(19) United States
(12) Patent Application Publication
(10) Pub. No.: US 2012/0238661 A1
(43) Pub. Date: Sep. 20, 2012

(54) Processes for the Synthesis of Compounds from Cyclic Carbonates

(75) Inventors: Ramesh Iyer, Harleysville, PA (US); Peter McKenna, Suffolk (GB); Mark Smallridge, Hythe (GB); Melissa Matthews, Southampton (GB); Anbazhagan Natesh, North Wales, PA (US)

(73) Assignee: Cognis IP Management GmbH, Dusseldorf (DE)

(21) Appl. No.: 13/349,630

(22) Filed: Jan. 13, 2012

Related U.S. Application Data
(60) Provisional application No. 61/432,632, filed on Jan. 14, 2011.

(51) Int. Cl. C07C 269/04; C08L 33/26; C07C 271/10; C07F 7/10

(52) U.S. Cl. 523/106; 560/157; 556/420

(57) ABSTRACT

The invention provides compounds containing the ring-opening products of ethylenically unsaturated cyclic carbonates for use in preparing hydrogel polymers, as well as methods for their preparation. A preferred method of the invention includes the reaction of nucleophilic compounds containing Si, F, N or O atoms, or combinations thereof, or latent moieties in the form of unsaturated or hydroxylated functional groups, with ethylenically unsaturated glycerol carbonate derivatives. The compounds and hydrogel polymers are useful for the preparation of contact lenses.

### FIELD OF THE INVENTION

The invention is related to processes for the synthesis of compounds, including certain novel compounds, based upon the reaction of cyclic carbonates, and more particularly to such reactions and reaction products having utility in the formation of monomers, macromers, oligomers and polymeric materials useful for the formation of hydrogels. The invention also relates to the use of such materials in the fields of hydrogel contact lenses, wound healing, controlled drug delivery, medical devices, catheters, stents and tissue engineering.

### BACKGROUND OF THE INVENTION

A hydrogel is a hydratable crosslinked polymeric system. Hydrogels useful in many applications are also oxygen permeable and bio-compatible, making them preferred materials for producing bio-medical devices and, in particular, contact or intraocular lenses. Conventional hydrogels are prepared from monomer mixtures predominantly containing hydrophilic monomers such as 2-hydroxyethyl methacrylate (HEMA) or N-vinyl pyrrolidinone (NVP), and hydrophobic monomers or macromers to obtain a polymer having a required hydration capacity and oxygen permeability. The oxygen permeability is commonly associated with polymers found from hydrophobic monomers containing siloxane or fluoro polymer moieties. U.S. Pat. Nos. 4,495,313, 4,889,664 and 5,039,459 disclose the formation of conventional hydrogels. The oxygen permeability of such conventional hydrogel materials are related to water content of the materials, and is typically below a Dk value of 20-30. For contact lenses made of the conventional hydrogel materials, the level of oxygen permeability is suitable for short-term wear of the contact lenses; however, that level of oxygen permeability may be insufficient to maintain a healthy cornea during long-term wear of the contact lenses (e.g., 30 days without removal). Efforts have been made and continue to be made to increase the oxygen permeability, and water content or hydration of conventional hydrogels, without adversely affecting the physical properties of the hydrogel polymers.

One way to increase the oxygen permeability of hydrogels is to add silicone-containing monomers or macromers, and/or fluorine-containing monomers or macromers to the hydrogel formulation to produce the hydrogels. Silicone-containing hydrogels generally have higher oxygen permeabilities than conventional hydrogels. Silicone-containing hydrogels have typically been prepared by polymerizing mixtures containing at least one organic silicone-containing monomer and at least one hydrophilic monomer. Either the silicone-containing or the hydrophilic monomer may function as a crosslinking agent (a crosslinking agent is a monomer having multiple polymerizable functionalities), or a separate crosslinking agent may be employed.

The formation of silicone hydrogels has been disclosed in U.S. Pat. Nos. 4,711,943, 4,954,587, 5,010,141, 5,079,319, 5,115,056, 5,260,000, 5,336,797, 5,358,995, 5,387,632, 5,451,617, 5,486,579, 5,789,461, 5,776,999, 5,760,100, 5,849,811 and WO 96/31792, the contents of which references are incorporated herein by reference.

U.S. Pat. No. 3,808,178 discloses the formation of co-polymers with low molecular weight silicone-containing monomers and various hydrophilic monomers. U.S. Pat. No. 5,034,461 describes silicone-containing hydrogels prepared from various combinations of silicone-polyurethane macromers and hydrophilic monomers such as HEMA, N-vinyl pyrrolidinone (NVP) and/or dimethylacrylamide (DMA). The addition of methacryloyloxypropyltris(trimethylsiloxy)silane (TRIS) reduced the modulus of such hydrogels, but in many examples the modulus was still higher than may be required.

U.S. Pat. Nos. 5,358,995 and 5,387,632 describe hydrogels made from various combinations of silicone macromers, TRIS, NVP and DMA. Replacing a substantial portion of the silicone macromer with TRIS reduced the modulus of the resulting hydrogels. The two publications from the same author, “The Role of Bulky Polysiloxane Alkylmethacrylates in Polyurethane-Polysiloxane Hydrogels”, J. Appl. Poly. Sci., Vol. 60, 1193-1198 (1996), and “The Role of Bulky Polysiloxanyl Alkylmethacrylates in Oxygen Permeable Hydrogel Materials”, J. Appl. Poly. Sci., Vol. 56, 317-324 (1995) also describe experimental results indicating that the modulus of hydrogels made from mixtures of silicone-macromers and hydrophilic monomers such as DMA, decreases with added TRIS.

The use of methacryloyloxypropyl tris(trimethylsiloxy)silane (TRIS) to make hard contact lenses was described in WO 91/10155 and in JP 61123609. The use of methacryloylpropyl polydimethylsilanes has been disclosed in EP 0 940 693 as useful soft contact lens monomers. U.S. Pat. No. 4,711,943 discloses N-[(tris(trimethylsilyloxy)silylpropyl]methacryloyloxyglyceryl carbamate (alternatively, 3-({[2-hydroxy-3-(methacryloyloxy)propoxy]carbonyl}amino)propyl tris(trimethylsiloxy)silane) as a hydrophilic monomer for contact lens materials. Other carbamate-containing monomers for contact lenses are disclosed in WO 2010/102747 (by applicants), US 2006/0063852, and EP 0 819 258.

Notwithstanding some degree of success in connection with prior synthesis methods and the resulting reaction products, particularly monomers, macromers and oligomers useful in the formation of hydrogels, applicants have come to recognize a continuing need for new synthesis methods and for compounds, compositions, materials, and products resulting therefrom. With respect to hydrogels, applicants have come to appreciate the need for synthesis methods and the resulting products which are advantageous in the formation of hydrogels which possess a complex set of properties, including softness, high oxygen permeability, suitable water content (hydration), surface wettability, and sufficient elasticity while remaining tear-resistant (high tensile strength). None of the above-cited references discloses the synthesis of such monomers by the novel method of reacting an appropriately substituted cyclic carbonate with an appropriately functionalized nucleophile, as now disclosed in the present invention.

### BRIEF SUMMARY OF THE INVENTION

Applicants have developed new synthesis methods for desirable monomers for contact lens construction, based on the reaction of compounds comprising a cyclic carbonate moiety with at least one nucleophilic compound. The ring-opened products may also be further elaborated to introduce required functionality, such as silicon- or fluorine-containing groups.

In preferred embodiments, the synthesis methods of the present invention comprise the step of ring-opening a cyclic carbonate-containing compound, preferably such a compound in the form of a glycerol carbonate containing at least one double bond capable of participating in a polymerization reaction.

In preferred embodiments, the synthesis methods comprise providing at least one compound according to Formula (I) comprising a cyclic carbonate moiety:

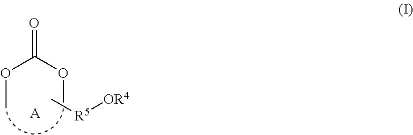

and reacting such compound with a nucleophilic compound in accordance Formula (II):

[H−Y]ₙ—X (II)
where n=1-3
   to produce at least one reaction product in accordance with Formula (III):

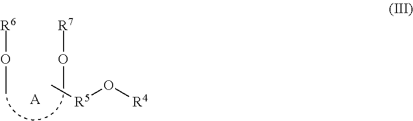

where
A represents a substituted or unsubstituted, saturated or unsaturated, mono-, bi- or tri-cyclic ring-closure group having from 3 to 12 carbon atoms, preferably from about 3 to 9 carbon atoms and even more preferably from 3 to 6 carbon atoms;
where R⁶ and R⁷ are different and each is either H or a monovalent group corresponding to X—Y—C(═O)—, where n=1;
R⁴ is a substituted or unsubstituted, branched or unbranched mono-valent group containing at least one carbon-carbon double bond and having from 2 to about 12 carbon atoms, preferably from about 2 to 6 carbon atoms and even more preferably from 2 to 4 carbon atoms;
R⁵ is a substituted or unsubstituted, saturated or unsaturated, branched or unbranched bivalent group having from 1 to about 6 carbon atoms, preferably from about 1 to 5 carbon atoms and even more preferably from 1 to 3 carbon atoms;
Y is a divalent or trivalent moiety selected from the group consisting of —O—, —S—, —NH—, —N(CH3)-, —N(C2H5)- and —N(R)—, where R is linear or branched or cyclic, saturated or unsaturated C3-C12 alkyl, optionally substituted with one or more hydroxy, C1-C4 alkoxy, C1-C4 haloalkoxy, or halogen groups; preferred Y groups are amines;

and
X is a group containing one or more carbon atoms and at least one atom selected from the group consisting of Si, F, O, N and combinations of any two or more of these; preferred X groups contain silicon and oxygen, and/or fluorine. Alternatively, X may be a group containing latent functionality which allows subsequent elaboration to install the desired Si-, F-, O-, and/or N-containing groups. Preferred latent functionality includes alkenes, alkynes and hydroxy groups.

As used herein, the term “ring-closure group” refers to any combination of covalent bonds and/or atoms that serve to, either directly or indirectly, covalently connect the carbon atoms in the structure of Formula (I) to form a cyclic carbonate structure.

As used herein, the term “substituted or unsubstituted” means that each carbon atom in the group may be functionalized with only hydrogen atoms, or may have one or more carbon-hydrogen bonds substituted by any one or more of carbon-halogen bonds, carbon-carbon bonds, carbon-nitrogen bonds, carbon-oxygen bonds, carbon-silicon bonds, and the like.

As used herein, the term “saturated or unsaturated” means that any two of the carbon atoms in the group may be bound to one another by a single bond, a double bond, or a triple bond.

### DETAILED DESCRIPTION OF PREFERRED EMBODIMENTS

Applicants contemplate that, in view of the teachings contained herein, those skilled in the art will be able to adapt a wide variety of particular compounds in accordance with each of Formulas (I) and (II) to produce a wide variety of reaction products in accordance with Formula (III) using a wide range of process conditions and parameters, including reaction temperatures, reaction pressures, reaction times, catalysts, and the like. In general, it is contemplated that the reaction will proceed in preferred embodiments under relative low temperature, exothermic conditions.

With respect to Formula (I) compounds, it is contemplated that each of the following compounds in accordance with Formulas (IA)-(ID) are adaptable for use in connection with the present invention:

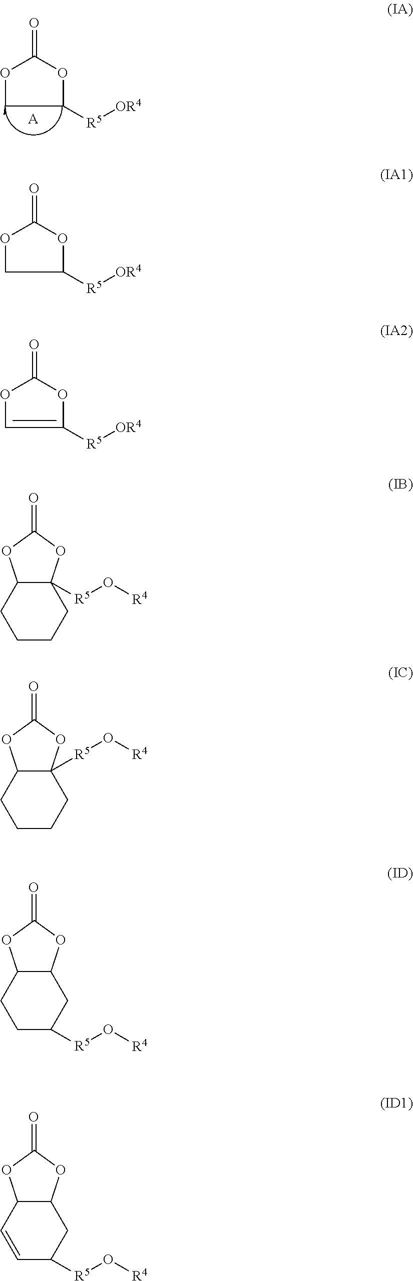

wherein R⁵ and R⁴ are as defined above. In certain highly preferred embodiments, the synthesis reaction of the present invention comprises providing a compound in accordance with one of Formula (IA), and even more preferably Formula (IA1).

In certain highly preferred embodiments, R⁴ is a group containing an acrylate or methacrylate functionality, and even more preferably in certain embodiments R⁴ is selected from

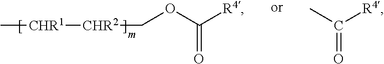

where
R^{4′} is a monovalent group

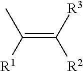

where R¹, R² and R³ are each independently H or —CH₃, and
m is from 1 to 20.

A preferred aspect of the present invention is illustrated below in connection with the following reaction scheme:

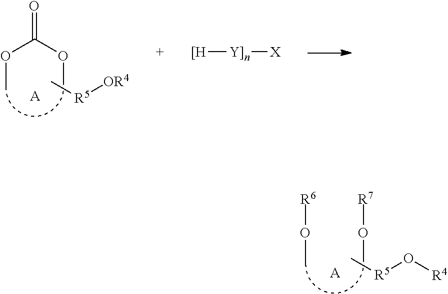

where n is 1-3; and
A, X, Y and each of the R groups is as defined above, it being recalled that at least one of R⁶ or R⁷ is a monovalent group X—Y—C(═O)—, where n=1.

Alternatively, cyclic carbonates of Formula (I) may be reacted with nucleophilic compounds of Formula (II), wherein Y is a nucleophilic atom or group, and X contains a latent functional group which may be further reacted to introduce the desired atom or atoms selected from the group consisting of Si, F, O, N and combinations thereof.

Thus, for example, glycerol carbonate methacrylate (GCMA) may be reacted with allylamine, 3-aminopropyne, ethanolamine, 3-aminopropanol, and the like, to give an intermediate (III) in which R⁴ contains alkene, alkyne or hydroxy functionality. This latent functionality may be further elaborated to add the atoms or groups which impart the desired characteristics to the polymers and hydrogels of the invention.

As a specific example, glycerol carbonate methacrylate (GCMA) may be reacted with allylamine to provide allylaminocarbonyloxy-(hydroxy)propyl methacrylate, which may be further reacted with a silane under hydrosilylation conditions to form the desired silicon-containing monomer (see Examples 5-7). Preferred silanes are selected from the group consisting of tris(trimethylsilyloxy)silane, bis(trimethylsilyloxy)(methyl)silane, and n-butyl(dimethyl)silyl(polydimethylsilyloxy)dimethylsilane.

Applicants have come to appreciate that the preferred synthesis processes of the present invention permit the formation of molecules having a difficult-to-obtain but highly advantageous combination of properties, preferably that will be manifest in the polymeric material formed thereby. This flexibility and advantage stems, at least in substantial part, from the ability to form such compounds in accordance with Formula (III) with numerous and varied X groups.

In accordance with certain preferred embodiments, X is a monovalent group selected from:

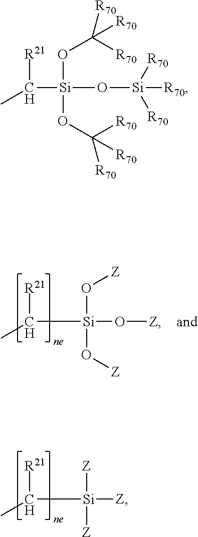

and/or contains one or more bivalent groups selected from
—O—, —NH—, —[CF₂]_{b}—, —[C(R²²)₂]_{b}—, R^{55B} R^{55C},

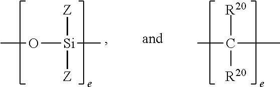

where

each R²⁰ is independently H or F

each R²¹ is independently H, a C1-C4 alkyl group, or R²³

each R²² is independently selected from H and a halogen, preferably F,

and

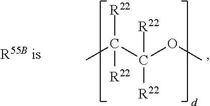

and
R^{55C} is

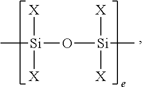

where each b is independently 1 to 50,
each d is independently from 1 to 50, and
e is from 1 to 100, more preferably in certain embodiments from 1 to 50, and from 1 to about 30 is certain preferred embodiments,

and where
R⁷⁰ is H or a straight chain or branched, substituted or unsubstituted C1-C4 alkyl group, and in certain preferred embodiments methyl,
each R²¹ is independently H, a C1-C4 alkyl group, or R²³,
where
R²³ is R²⁵—O—(CR^{25A}H—CR^{25A}HO)ₓ—CHR^{25A}CR^{25A}H—,
   where
   each R²⁵ is independently a straight chain or branched, substituted or unsubstituted, C1-C4 alkyl group,
   each R^{25A} is independently H, a straight chain or branched, substituted or unsubstituted, C1-C4 alkyl group
   and x is from about 1 to about 50,
where
each X is as defined above,
each Z is independently H, an alkyl or haloalkyl moiety having from 1 to about 10 carbon atoms, with and without ether linkages between carbon atoms, or a siloxane group corresponding to —O—Si—R⁹, with each R⁹ being independently a straight chain or branched,
substituted or unsubstituted C1-C4 alkyl group, or a phenyl group, and

where
each ne is independently from 1 to 4.

More preferably cyclic carbonates of Formula (IA3) are reacted with amines of Formula (IIA) to provide monomers of Formula (IIIA):

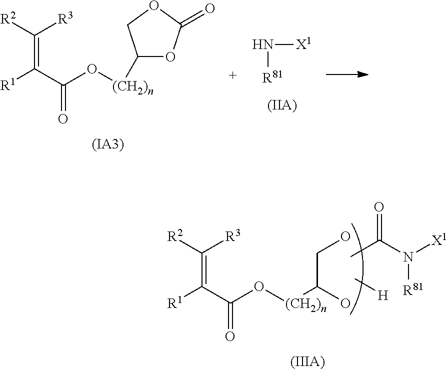

wherein R¹, R² and R³ are independently H or methyl;
n=1-12
R⁸¹ is hydrogen, C₁-C₁₂ alkyl optionally substituted with one or more hydroxy, C₁-C₄ alkoxy, halogen or C₁-C₄ haloalkoxy groups;
X¹ is (CH₂)ₙR⁸², where
n=1-12, and
R⁸² is haloalkyl, SiR⁸³₃, OSiR⁸³₃, or heterocyclyl,
where R⁸³ are independently C₁-C₁₂ alkyl, C₁-C₆ alkoxy, trimethylsilyloxy (OTMS), C₃-C₆ cycloalkyl, C₃-C₆ cycloalkoxy, [OSi(Me)₂]_{z}(CH₂)ₙNH₂, [OSi(Me)₂]_{z}OSiR⁸⁵₃, where z=1-1000, and R⁸⁵ is independently C₁-C₁₂ alkyl,

or
aryl or heteroaryl, optionally substituted with hydroxy, C₁-C₄ alkyl, C₁-C₄ alkoxy, halogen, C₁-C₄ hydroxyalkyl, C₁-C₄-alkoxyl-C₁-C₄-alkyl; and
where heterocyclyl includes saturated or partially unsaturated heterocycles, such as, without limitation,

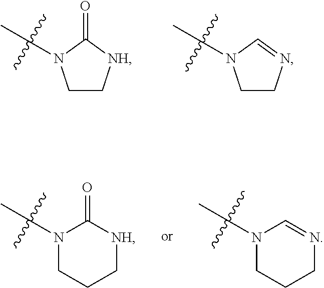

Alternatively, for the reaction where the amine is of Formula (IIB):

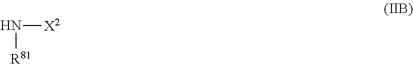

where X² is (CH₂)ₙR⁸⁴, where n=1-12, and

R⁸⁴ is C₂-C₁₂ alkenyl, C₂-C₁₂ alkynyl, C₁-C₁₂ hydroxyalkyl, or C₂-C₁₂ hydroxyhaloalkyl;

to form monomers of Formula (IIIB):

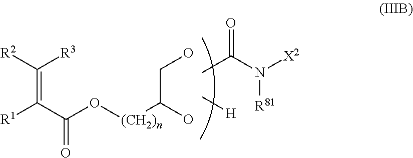

R⁸⁴ is further elaborated to introduce silicon- and/or fluorine-containing functionality,

Preferred amines (IIA) are selected from the group consisting of 2,2,2-trifluoroethylamine; 3-tris(trimethylsilyloxy)silylpropylamine; 3-bis(trimethylsilyloxy)-(methyl)silylpropylamine; n-butyl(dimethyl)silyl-(polydimethylsilyloxy)propyl amine, with z=1-1000; 1,3-bis-(3-aminopropyl)-1,1,3,3-tetramethyldisiloxane; bis-(3-aminopropyl)-poly(dimethylsiloxane), z=1-1000; and 1-(2-aminoethyl)-imidazolidin-2-one.

Preferred amines (IIB) include allylamine.

One example of the elaboration of an analog where R⁸⁴ is alkenyl is provided below (see Examples 4 and 5).

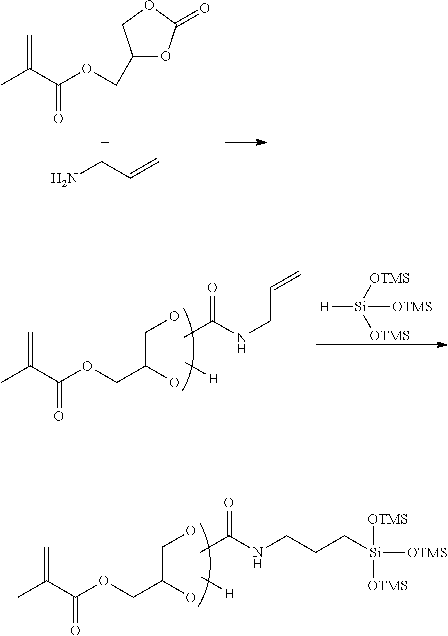

Another aspect of the present invention comprises the novel products of the processes, including those of Formulae (III) and (IIIA), excluding the known 3-({[2-hydroxy-3-(methacryloyloxy)propoxy]carbonyl}amino)propyl tris(trimethylsiloxy)silane.

### Glycerol Carbonate Derivatives

The monomers, oligomers and macromers of the invention most preferably comprise carbonate-ring-opening derivatives of glycerol carbonate containing an ethylenically unsaturated, polymerizable double bond. Cyclic carbonate compounds and their methods of preparation are disclosed in “Cyclic Carbonate Functional Polymers and Their Applications”, Dean C. Webster, Progress in Organic Coatings, 47, pages 77-86 (2003), and the references cited therein, as well as U.S. Pat. No. 5,763,622; the entire contents of both references being incorporated herein by reference. Ethylenically unsaturated glycerol carbonates can be readily prepared by reaction of CO₂ with glyceryl methacrylate, by transesterification of glycerol carbonate with methylmethacrylate, by reaction of glycerol carbonate with methacryloyl chloride, or by the reaction of glycerol carbonate chloroformate with methacrylic acid. Acrylated glycerol carbonate can be prepared by analogous methods. The ethylenically unsaturated glycerol carbonate is reacted with a compound containing one or more nucleophilic groups, such as amino, hydroxyl, or thiol groups, to form the compounds of the invention. Primary amines provide a rapid reaction and are preferred. The reaction is generally carried out at a temperature from about 0° C. to 200° C., depending on the reactivity of the nucleophile with the cyclic carbonate functional group of the ethylenically unsaturated glycerol carbonate.

Therefore, in accordance with certain most preferred aspects of the invention, the disclosed synthesis method is utilized to form one or more compounds as described below, usually obtained as an isomeric mixture. Thus,

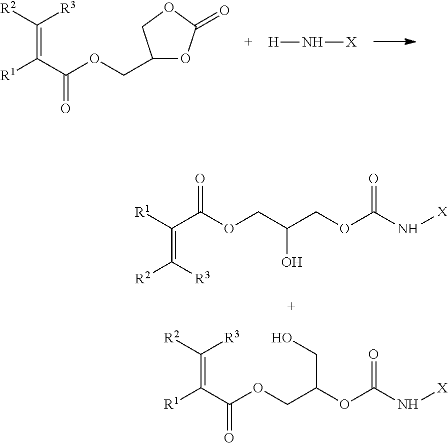

Possible additional isomer:

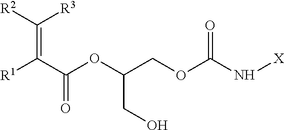

This isomer mixture may also be depicted as Formula (IIIC):

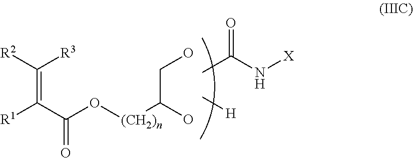

The reaction is regioselective, with the amine reacting at the cyclic carbonate carbonyl preferentially over Michael addition to the (meth)acrylate moiety.

Preferred compounds (IIIC) of the invention include:

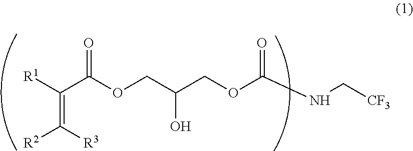

and its isomers, as above, wherein (B) is the residue of the ethylenically unsaturated glycerol carbonate and its isomers. (B) will be used to represent the glycerol carbonate residue in other preferred compounds of the invention, below.

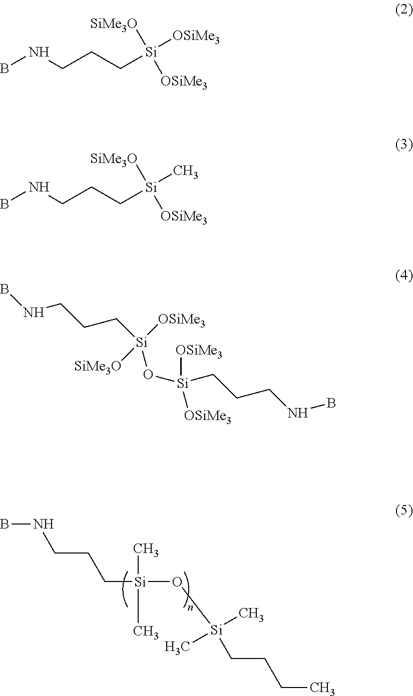

wherein n is a number of from 1 to 1,000

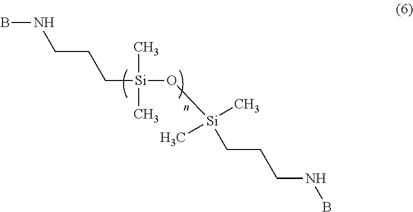

wherein n is a number of from 1 to 1,000

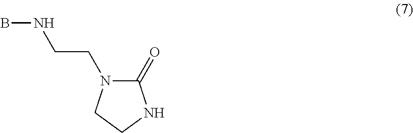

The compounds of the present invention can be monomers, oligomers or macromers. As used herein, the term “monomer” denotes an olefinically unsaturated small molecule which may be oligomerized or polymerized to form materials appropriate for contact lenses. Monomers of the invention preferably have a molecular weight of about 600 Daltons or less. Further, the term “oligomer” refers to compounds of the invention which preferably have a molecular weight (MW) of up to about 1,000 Daltons. The “macromers” of the invention have a molecular weight (MW) above about 1,000 up to about 50,000 Daltons, preferably about 1000 to 30,000 Daltons, most preferably about 1000-10,000 Daltons.

The compounds of the invention can be monofunctional or polyfunctional in relative to the ethylenically unsaturated group. The compounds, oligomers and macromers of the present invention include the reaction product of the ethylenically unsaturated glycerol carbonate with α,ω-amine terminated polyfluoronated compounds, α,ω-amine terminated polydimethyl siloxane, α,ω-hydroxy terminated polyfluorinated compound, α,ω-hydroxy terminated polydimethylsiloxanes oligomers, α,ω-thiol terminated polyfluorinated compounds, α,ω-thiol terminated polydimethylsiloxanes, α,ω-hydroxyl, amine or thiol terminated polyalkoxide wherein the alkoxide residue group contain 2 to 4 carbon atoms.

Further, the compounds of the invention contain substantially no analogs (less than or equal to 1% by weight) having unopened pendant carbonate rings since the nucleophile is attached to the carbonate residue through the opened carbonate ring. Any cyclic carbonate in the reaction mixture would be due to unreacted ethylenically unsaturated glycerol carbonate compounds.

The ethylenically unsaturated compounds of the invention can be used alone or in a mixture with other monomers, oligomers or macromers to form linear polymers with pendant residues of the nucleophile, or can be reacted with other ethylenically unsaturated polyfunctional components to form crosslinked polymers. In this manner hydrogels may be produced with the required balance of oxygen permeability, hydration properties, and physical properties suitable for a particular use. A polymerizable composition comprising from about 30-80% by weight of one or more compounds of the invention and about 0-20% by weight of one or more hydrophilic monomers is suitable for the preparation of polymers and hydrogels having the required properties for contact lenses.

The compounds of the invention can be used to form polymers with monomers and oligomers such as 2-hydroxyethyl methacrylate (HEMA), methacrylic acid (MA), dimethylacryamide (DMA), N-vinyl pyrrolidinone (NVP), methacryloyloxypropyltris(trimethylsiloxy)silane (TRIS), glycerol monomethacrylate (GMMA) and alkyloxy-1,2 propane diol. The compounds of the invention in admixture with other polymerizable components can be polymerized by free radical polymerization using thermal initiators such as alkyl or aryl peroxides, or by photoinitiation using, for example, UV radiation or electron beam radiation. The polymerization initiators or activators required are well known in the polymerization art. Electron beam radiation polymerization processes do not generally require other initiators or activators.

The processes and compounds set out above are for illustrative purposes only and are not intended to be limiting as to the monomers, oligomers or macromers which can be used to form useful hydrogels of the present invention. Further, the following Examples are representative of the invention, and in no way limit the scope thereof.

### EXAMPLES

Karstedt's catalyst is platinum divinyltetramethyldisiloxane (CAS No. 68478-92-2). MEHQ is 4-methoxyphenol, or O-methyl hydroquinone (CAS No. 150-76-5).

The glycerol carbonate methacrylate (GCMA) used in these reactions was prepared from glycerol carbonate by transesterification using zirconium acetylacetonate catalyst using the method described in Patent Application WO 2007/071470 by Schmitt, Knebel and Caspari of Röhm GmbH & Co. Purity as judged by GC was 95-96 Area %.

### Example 1

### 1,3-Bis[3-({[2-hydroxy-3-(methacryloyloxy)propoxy]carbonyl}amino)propyl]1,1,3,3-tetramethyldisiloxane

To glycerol carbonate methacrylate (1.0 g, 5.4 mmol) cooled in an ice/water bath was added 1,3-bis(3-aminopropyl)-1,1,3,3-tetramethyldisiloxane (0.67 cm³, 2.4 mmol) dropwise with stirring. The reaction mixture was stirred for 2 h in the ice/water bath and then left to stand at room temperature for 16 h to give a thick, colourless oil.

IR: (cm⁻¹) 3357 (OH), 2955 (alkyl), 1705 (C═O), 1638 (C═C), 1532 (amide II), 1251 (Si-Me) 1043 (Si—O) and 775 (Si-Me).

¹H NMR: (C²HCl₃, 400 MHz) δ_{H} 0.04 (12H, s, SiCH₃), 0.44-0.51 (4H, m, SiCH₂), 1.46-1.56 (4H, m, SiCH₂CH₂), 1.89-1.96 (6H, m, CCH₃), 3.08-3.16 (4H, m, CH₂N), 3.60-4.50 (10H, m, 2×CH₂O and CHOH), 5.56-5.60 (2H, m, CHH═C) and 6.08-6.16 (2H, m, CHH═C).

### Example 2

### Bis[3-({[2-hydroxy-3-(methacryloyl-oxy)propoxy]carbonyl}amino)propyl] Terminated poly(dimethyl siloxane)

To glycerol carbonate methacrylate (1.0 g, 5.4 mmol) cooled in an ice/water bath was added bis(3-aminopropyl) terminated poly(dimethylsiloxane) (6.2 cm³, 2.4 mmol) dropwise with stirring. The reaction mixture was stirred for 2 h in the ice/water bath and then left to stand at room temperature for 16 h to give a thin, colourless oil.

IR: (cm⁻¹) 2963 (alkyl), 1725 (C═O), 1533 (amide II), 1258 (Si-Me), 1010 (Si—O) and 787 (Si-Me).

¹H NMR: (C²HCl₃, 400 MHz) δ_{H} 0.04 (s, SiCH₃), 0.50-0.56 (4H, m, SiCH₂), 1.48-1.57 (4H, m, SiCH₂CH₂), 1.93-1.98 (6H, m, CCH₃), 3.12-3.19 (4H, m, CH₂N), 3.60-5.00 (10H, m, 2×CH₂O and CHOH), 5.58-5.67 (2H, m, CHH═C) and 6.11-6.17 (2H, m, CHH═C).

### Example 3

### 3-({[2-hydroxy-3-(methacryloyloxy)propoxy]carbonyl}amino)propyl tris(trimethylsiloxy)silane

3-Aminopropyl tris(trimethylsiloxy) silane (1.9 g, 5.4 mmol) was added dropwise to stirred GCMA (1.0 g, 5.4 mmol). The reaction mixture was stirred for 26 h and monitored by IR spectroscopy. The samples at 20.5 h and 26 h were identical, suggesting that the reaction had stopped, however a small carbonate signal (1802 cm⁻¹) suggested that some unreacted GCMA was still present.

A few drops of 3-aminopropyl tris(trimethylsiloxy) silane were added and the reaction mixture stirred until the C═O carbonate signal was reduced. The reaction product was obtained as a colourless oil (1.27 g, 43%).

IR: (cm⁻¹) 3375 (OH), 2958 (CH), 1718 (C═O ester and urethane), 1532 (amide II), 1250 (Si-Me), 1018 (Si—O) and 836 (Si-Me).

¹H-NMR(C²HCl₃, 400 MHz) 0.09 (s, SiCH₃), 0.41-0.45 (2H, m SiCH₂), 1.48-1.56 (2H, m, SiCH₂CH₂), 1.94 (3H, s, CCH₃), 3.12-3.17 (2H, m CH₂NH), 3.72-5.06 (5H, m, 2×CH₂O and CHOH)

5.58-5.59 (1H, m, one of C═CH₂) and 6.12-6.16 (1H, m, one of C═CH₂).

Inspection of the spectra suggested that the reaction had been successful and that the product is present as a mixture of isomers. It also appeared that some Michael addition had occurred, as judged by small signals at 1.2 and 2.5-3.0 ppm.

### Example 4

### Ring Opening GCMA with Allylamine

Allylamine (40 cm³, 0.53 mol) was added to GCMA (100.51 g, 0.54 mol, J Jones). The reaction mixture increased in temperature and was therefore cooled in an ice/water bath in order to limit the temperature increase (max temp observed 66° C.). Once the temperature had cooled to 30° C. the ice/water bath was removed and the reaction mixture allowed to cool to room temperature and stirred for 4 days. GC showed GCMA remaining in the reaction mixture. Further allylamine (2 cm³) was added and the reaction mixture stirred for 2 days. GC showed GCMA remaining in the reaction mixture. Further allylamine (2 cm³) was added and the reaction mixture stirred for 1 day and then transferred to a sample jar (130.66 g, 100%). GCMS showed the presence of allylamine, GCMA and the desired product.

GC: Instrument 2. Sample preparation ca. 3 drops in acetone.

GCMA 4.49 Area %; Intermediate 78.51 Area %.

The above allylaminocarbonyloxy hydroxypropyl methacrylate (107.28 g) was mixed with stripped BREOX® 60W1000 carrier oil (15.12 g) and dosed with MEHQ (36.4 mg, 339 ppm). The resulting mixture was stripped using the short path wiped film (SPWF) evaporator using the diffusion pump in conjunction with the rotary vane pump (chiller 5° C., heater 60° C., vacuum 0.004-0.009 mbar). A pale yellow-green distillate was obtained (3.8 g, 4%) which was a mixture of GCMA and the desired product as judged by GCMS (Rt 11.83 min GCMA, 15.19 min allylaminocarbonyloxy hydroxypropyl methacrylate).

The orange-brown residue (103.62 g) was reintroduced into the SPWF and distilled using the diffusion pump in conjunction with the rotary vane pump (chiller 5° C., heater 80-95° C., vacuum 0.003 mbar). Allylaminocarbonyloxy hydroxypropyl methacrylate distilled as a yellow oil (43.48 g, 41%).

IR: 3359 (OH), 2929 (OH), 1698 (C═O ester), 1638 (C═C), 1528 (amide II) and 1159 (C—O).

GC/MS: Method MLM Scan General AS-Spilt. Sample diluted with acetone (3 drops sample/GC vial). Rt 15.32 min and 15.43 min. Double peak possibly due to product isomers.

¹H-NMR: Consistent with desired product. Triplet corresponding to GCMA present in spectrum at 4.57 ppm.

### Example 5

### Hydrosilylation of Allylaminocarbonyloxyhydroxypropyl Methacrylate with Tris(Trimethylsiloxysilane) Using Karstedt's Catalyst

To a round bottom flask fitted with a condenser and thermometer was added tris(trimethylsiloxy)silane (2.03 g, 0.0069 mol), toluene (5 cm³), allylaminocarbonyloxyhydroxypropyl methacrylate (2.00 g, 0.0082 mol) and Karstedt's catalyst (0.2 cm³). The reaction mixture was heated using an oil bath (set point 80° C.). After a reaction time of 1 h a sample was removed and analysed by IR. A Si—H signal (2201 cm⁻¹) remained and so further catalyst (0.2 cm³) was added to the reaction mixture. After a total reaction time of 2 h the reaction was deemed to be complete by IR spectroscopy and so the reaction mixture was allowed to cool to room temperature. The reaction mixture was slurried with silica for ca. 10 min, filtered and then concentrated under reduced pressure to give a yellow-orange oil (2.73 g, 73%).

IR: 3357 (OH), 2959 (CH), 1705 (C═O), 1639 (C═C), 1528 (amide II), 1250 (Si-Me), 1044 (Si—O) and 836 (Si-Me).

GC: Instrument 11. Sample preparation 2 drops in hexane.

Rt 18.32 min 65.41 Area %. Note methacrylate starting material not visible by this method.

The above compound (11.89 g, 300 ppm MEHQ) was diluted with hexane (50 cm³) and the orange solution transferred to a separating funnel. The solution was washed with aqueous sodium hydroxide solution (2×50 cm³, 0.5 M) and brine (2×50 cm³). The organic layer was concentrated under reduced pressure to give an orange oil (10.12 g, 85%, 2 ppm MEHQ), identical with the material prepared in Example 3.

### Example 6

### Hydrosilylation Using Bis(Trimethylsiloxy)Methylsilane

Allylaminocarbonyloxyhydroxypropylmethacrylate (20.00 g, 0.082 mol, distilled), bis(trimethylsiloxy)methylsilane (15.26 g, 0.069 mol, Gelest SIB1844.0), toluene (50 cm³) and Karstedt's catalyst (2 cm³) were charged to a 3 necked flask fitted with a thermometer and condenser. The reaction mixture was stirred and heated to 80° C. for 1 h. A sample was removed and analysed by IR and GCMS; which showed no silane remaining. The yellow reaction mixture was allowed to cool to room temperature, and then concentrated under reduced pressure to give a yellow liquid (33.34 g, >100%).

Analysis: Reaction Sample 1 h

IR: 3418 (OH), 2958 (CH), 1722 (C═O), 1252 (Si-Me), 1043 (Si—O) and 841 (Si-Me).

GCMS: (MLM Scan, General AS Split)

4.55 min: Toluene

24.85 min: m/z 39, 69 [CH₃C═CH₂C═O]⁺, and 133.

The above compound (29.19 g, J Baker) was mixed with stripped BREOX® 60W1000 carrier oil (6.99 g) and MEHQ (0.1154 g, 4000 ppm) and the mixture stirred until all solids had dissolved. The mixture was then stripped using the SPWF evaporator (chiller 5° C., heater 60° C., vacuum 0.045-0.048 mbar). A distillate was obtained (0.22 g) along with a residue (25.22 g). The residue was reintroduced to the SPWF evaporator and distilled (chiller 10° C., heater 110° C., vacuum 0.043-0.044 mbar). A pale yellow distillate was obtained (9.12 g, 31%) along with a yellow residue (14.56 g).

IR: (Distillate) 3358 (OH), 2958 (CH), 1702 (C═O), 1639 (C═C), 1529 (amide II), 1251 (Si-Me), 1040 (Si—O) and 839 (Si-Me).

The above compound (7.52 g, Distilled) was diluted with hexane (50 cm³) and washed with sodium carbonate solution (2×50 cm³, 0.5 M) and saturated aqueous sodium chloride solution (2×50 cm³). The organic phase was concentrated under reduced pressure to give a yellow oil which contained some polymer (weight oil and polymer 3.49 g, 46%).

### Example 7

### Hydrosilylation, Using Monohydride Terminated Butyl-PDMS

Allylaminocarbonyloxyhydroxypropylmethacrylate (10.00 g, 0.041 mol, distilled), monohydride terminated PDMS (29.16 g, 0.034 mol, Gelest MCR-H07), toluene (25 cm³) and Karstedt's catalyst (1 cm³) were charged to a 3 necked flask fitted with a thermometer and condenser. The reaction mixture was stirred and heated to 80° C. for 1 h. A sample was removed and analysed by IR; which showed no silane remaining. The reaction mixture was allowed to cool to room temperature, and then concentrated under reduced pressure to give a yellow liquid (37.21 g, 100%).

Analysis: Reaction Sample 1 h

IR: 3416 (OH), 2961 (CH), 1724 (C═O), 1259 (Si-Me), 1015 (Si—O) and 792 (Si-Me).

The above product (ca. 35 g) was diluted with hexane (50 cm³) and slurried with silica (2.02 g) for 30 min. The silica was removed by gravity filtration through a Whatman No. 1 filter paper, and the filtrate concentrated under reduced pressure to give a yellow oil (28.76 g). MEHQ (2.0 mg, Rhodia) was added and the suspension stirred until the inhibitor had dissolved.

MEHQ: 156 ppm by GC

### Example 8

### Preparation of Tinted High-Water-Content HEMA-Based Contact Lenses; Comparison Example

A high-water formulation was made up using the formula detailed below in Table 1. The lenses were cured, and hydrated using commercially available saline to produce a stable hydrogel.

TABLE 1

| Material | Composition |
|---|---|
| Hydroxyethyl methacrylate | 96.44% |
| Methacrylic acid | 2.00% |
| PLURONIC ® F-127 | 1.00% |
| Ethylene glycol dimethacrylate | 0.34% |
| Benzoin methyl ether (UV initiator) | 0.17% |
| BISOMER IMT BLUE ® (Cognis Tint) | 500 ppm |

The formulation retained the dye in the polymer without leaching out into the saline solution.

### Example 9

### Preparation of Tinted Silicone Hydrogel-Based Contact Lenses

A silicone hydrogel formulation of the invention was made up using the formula detailed below in Table 2. The lenses were cured, and hydrated using commercial available saline to produce a stable hydrogel.

TABLE 2

| Material | Composition |
|---|---|
| Dimethacrylamide | 39.21% |
| Monomers (IIIC) | 55.32% |
| N-methyl pyrrolidinone (solvent) | 4.04% |
| Ethylene glycol dimethacrylate | 1.01% |
| Benzoin methyl ether | 0.41% |
| BISOMER IMT BLUE ® | 500 ppm |

The formulation retained the dye in the polymer without leaching out into the saline solution.

What is claimed is:

1. A method of preparing a monomer, macromer or oligomer comprising the steps of:
(a) providing a cyclic carbonate of Formula (IA3): 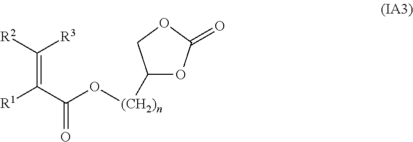
wherein R¹, R² and R³ are independently H or methyl, and n=1-12;
(b) adding, with control of temperature, an amine of formula (IIA): 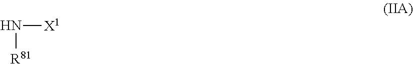
wherein R⁸¹ is hydrogen or C₁-C₁₂ alkyl optionally substituted with one or more hydroxy, C₁-C₄ alkoxy, halogen or C₁-C₄ haloalkoxy groups;
X¹ is (CH₂)ₙR⁸², wherein n=1-12, and
R⁸² is haloalkyl, SiR⁸³₃, OSiR⁸³₃, or heterocyclyl,
wherein R⁸³ are independently C₁-C₁₂ alkyl, C₁-C₆ alkoxy, trimethylsilyloxy (OTMS), C₃-C₆ cycloalkyl, C₃-C₆ cycloalkoxy, [OSi(Me)₂]_{z}(CH₂)ₙNH₂, [OSi(Me)₂]_{z}OSi R⁸⁵₃, where z=1-1000, and R⁸⁵ is independently C₁-C₁₂ alkyl,
or
aryl or heteroaryl, optionally substituted with hydroxy, C₁-C₄ alkyl, C₁-C₄ alkoxy, halogen, C₁-C₄ hydroxyalkyl, C₁-C₄-alkoxyl-C₁-C₄-alkyl; and
wherein heterocyclyl is selected from the group consisting of 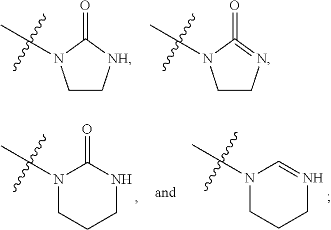
to form a ring-opened monomer of Formula (IIIA): 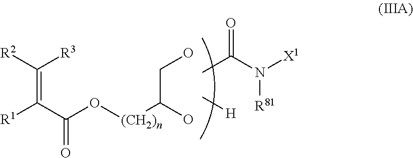
(c) optionally, distilling said monomer;
(d) optionally, further reacting said ring-opened monomer, optionally in the presence of a catalyst, to introduce functionality comprising Si, F, N, and/or O atoms; and
(e) optionally, removing said catalyst and/or distilling.

2. A method of preparing a monomer, macromer or oligomer comprising the steps of:
(a) providing a cyclic carbonate of Formula (IA3): 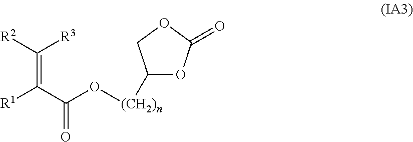
wherein R¹, R² and R³ are independently H or methyl, and n=1-12;
(b) adding, with control of temperature, an amine of formula (IIB): 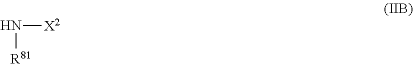
wherein R⁸¹ is hydrogen or C₁-C₁₂ alkyl optionally substituted with one or more hydroxy, C₁-C₄ alkoxy, halogen or C₁-C₄ haloalkoxy groups;
X² is (CH₂)ₙR⁸⁴, where n=1-12, and
R⁸⁴ is C₂-C₁₂ alkenyl, C₂-C₁₂ alkynyl, C₁-C₁₂ hydroxyalkyl, or
C₂-C₁₂ hydroxyhaloalkyl;
to form a ring-opened monomer of Formula (IIIB): 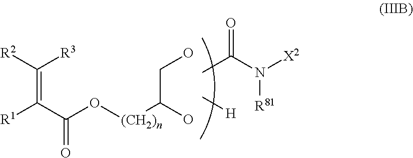
(c) optionally, distilling said monomer;
(d) further reacting said ring-opened monomer, optionally in the presence of a catalyst, to introduce functionality comprising Si atoms; and
(e) optionally, removing said catalyst and/or distilling.

3. The method of claim 1, wherein said cyclic carbonate comprises glycerol carbonate methacrylate and/or glycerol carbonate acrylate.

4. The method of claim 1, wherein said amine is selected from the group consisting of 3-tris(trimethylsilyloxy)silylpropylamine; 3-bis(trimethylsilyloxy)-(methyl)silylpropylamine; n-butyl(dimethyl)silyl(polydimethylsilyloxy)propyl amine, z=1-1000; 1,3-bis-(3-aminopropyl)-1,1,3,3-tetramethyldisiloxane; bis-(3-aminopropyl)poly(dimethylsiloxane), z=1-1000; 1-(2-aminoethyl)-imidazolidin-2-one; and 2,2,2-trifluoroethylamine.

5. The method of claim 2, wherein said amine comprises allylamine.

6. The method of claim 5, wherein step (d) comprises hydrosilylation with a silane.

7. The method of claim 6, wherein said silane is selected from the group consisting of tris(trimethylsilyloxy)silane, bis(trimethylsilyloxy)(methyl)silane, and n-butyl(dimethyl)silyl(polydimethylsilyloxy)dimethylsilane.

8. The product of the process of claim 1, excluding 3-({[2-hydroxy-3-(methacryloyloxy)propoxy]carbonyl}amino)propyl tris(trimethylsiloxy)silane.

9. The product of the process of claim 2, excluding 3-({[2-hydroxy-3-(methacryloyloxy)propoxy]carbonyl}amino)propyl tris(trimethylsiloxy)silane.

10. A hydrogel polymer comprising residues of the compound of claim 1, excluding 3-({[2-hydroxy-3-(methacryloyloxy)propoxy]carbonyl}amino)propyl tris(trimethylsiloxy)silane.

11. A hydrogel polymer comprising residues of the compound of claim 2, excluding 3-({[2-hydroxy-3-(methacryloyloxy)propoxy]carbonyl}amino)propyl tris(trimethylsiloxy)silane.

12. A contact lens comprising the hydrogel polymer of claim 10.

13. A contact lens comprising the hydrogel polymer of claim 11.

14. The method of claim 2, wherein said cyclic carbonate comprises glycerol carbonate methacrylate and/or glycerol carbonate acrylate.
